SEQUENCE LISTING (10 sequences)

SEQ ID NO: 1 - DNA, 43 bp, synthetic construct
  source 1..43: mol_type = other DNA
gagaggaatgtacacatggaagaaaaagaaatactctggaacg

SEQ ID NO: 2 - DNA, 49 bp, synthetic construct
  source 1..49: mol_type = other DNA
tacgccaagctttcattactcataaggcttatcttgataaaaatagttc

SEQ ID NO: 3 - DNA, 72 bp, synthetic construct
  source 1..72: mol_type = other DNA
ggtggtggtggtaatggtggtggtggttctggtggtggtggttcctcataaggcttatcttgataaaaatag

SEQ ID NO: 4 - DNA, 71 bp, synthetic construct
  source 1..71: mol_type = other DNA
attaccaccaccaccattaccaccaccaccattacctcctccaccaatggctaaagccttgattacatatg

SEQ ID NO: 5 - DNA, 79 bp, synthetic construct
  source 1..79: mol_type = other DNA
gaggaggaggaggaagaggaggaggaggctcaggaggaggaggctctgaaacatggattttttccttgttcatataatc

SEQ ID NO: 6 - DNA, 76 bp, synthetic construct
  source 1..76: mol_type = other DNA
ttcctcctcctcctcttcctcctcctcctcttccaccaccaccaatgcgagaggatacaaaggtttatgatattac

SEQ ID NO: 7 - DNA, 45 bp, synthetic construct
  source 1..45: mol_type = other DNA
tacgccaagctttcattatttattttcaaaaagacttgttgagtg

SEQ ID NO: 8 - DNA, 21 bp, synthetic construct
  source 1..21: mol_type = other DNA
tgaaagcttggcgtaatcatg

SEQ ID NO: 9 - DNA, 27 bp, synthetic construct
  source 1..27: mol_type = other DNA
gtgtacattcctctcttacctataatg

SEQ ID NO: 10 - DNA, 18 bp, synthetic construct
  source 1..18: mol_type = other DNA
catcaccatcaccatcac

(19) United States
(12) Patent Application Publication
(10) Pub. No.: US 2025/0325009 A1
(43) Pub. Date: Oct. 23, 2025

(54) COLOR FORMER BASED ON FUSION ENZYME PRODUCING NITRIC OXIDE AND USE THEREOF

(71) Applicant: Hefei University of Technology, Hefei (CN)

(72) Inventors: Peijun LI, Hefei (CN); Baocai XU, Hefei (CN); Bingyu LI, Hefei (CN); Huiting LUO, Hefei (CN); Kezhou CAI, Hefei (CN); Hui ZHOU, Hefei (CN); Qiong PAN, Hefei (CN); Jiaying QIN, Hefei (CN)

(73) Assignee: Hefei University of Technology, Hefei (CN)

(21) Appl. No.: 18/806,749

(22) Filed: Aug. 16, 2024

(30) Foreign Application Priority Data
Apr. 19, 2024 (CN) 2024104773801

(51) Int. Cl. A23L 13/40; C07K 19/00; C12N 1/20; C12N 9/02; C12N 9/06; C12N 15/75; C12R 1/125; C12R 1/19

(52) U.S. Cl. CPC A23L 13/48; C07K 19/00; C12N 1/20; C12N 9/0028; C12N 9/0075; C12N 15/75; C12R 2001/125; C12R 2001/19; C12Y 105/0103

(57) ABSTRACT

A color former based on a fusion enzyme producing nitric oxide and use thereof are provided. The color former includes the fusion enzyme producing nitric oxide. The fusion enzyme is formed by sequentially combining nitric oxide synthase, flavoprotein and flavoprotein reductase pairwise via linker peptides. The fusion enzyme provided by the present disclosure has relatively high enzyme activity, can catalytically produce a large amount of nitric oxide, and can effectively bind to myoglobin in meat products to produce nitrosylmyoglobin, thereby effectively enhancing the red color of the meat products to obtain a color forming effect equivalent to that of sodium nitrite, and providing a highly practical solution for nitrite color forming replacement of meat products and improvement of meat product safety.

### CROSS REFERENCE TO THE RELATED APPLICATIONS

This application is based upon and claims priority to Chinese Patent Application No. 2024104773801, filed on Apr. 19, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBNJLF080_SequenceListing.xml, created on Jul. 18, 2024, and is 10,028 bytes in size.

### TECHNICAL FIELD

The present disclosure belongs to the technical field of meat science, and particularly relates to a color former based on a fusion enzyme producing nitric oxide and use thereof.

### BACKGROUND

The color formation of fermented sausage depends on the addition of nitrite, and its reduction product nitric oxide (NO) interacts with myoglobin in meat to form a red pigment-nitrosylmyoglobin. However, due to teratogenicity and carcinogenesis potential of nitrite, its use in meat has received much attention. In order to reduce the use of nitrite in meat, it is crucial to seek its function alternative methods. In recent years, people make researches to the color forming effect of coagulase-negative Staphylococcus (CNS) in fermented meat products, where bacterial nitric oxide synthase can transform L-arginine into NO and L-citrulline, thereby promoting the formation of nitrosylmyoglobin in meat products.

In numerous alternative methods for color formation of nitrite, a microbial fermentation method has a huge potential. The patent application CN110800913A discloses a color former for replacing nitrite in processed meat products. One of the color formers is a bacterial powder or bacterial suspension of coagulase-negative Staphylococcus. When in use, its inoculation amount in meat product processing is 10⁶ CFU/g-10⁷ CFU/g meat. Another color former is composed of a bacterial powder or bacterial suspension of coagulase-negative Staphylococcus and L-arginine. When in use, the inoculation amount of the coagulase-negative Staphylococcus in meat product processing is 10⁶ CFU/g-10⁷ CFU/g meat, and the addition amount of L-arginine in meat product processing is 0.6%-1.2% of the mass of the meat product. The bacterial powder or bacterial suspension is prepared by culturing coagulase-negative Staphylococcus in a liquid culture medium. The patent application CN110800913A discloses that the use of coagulase-negative Staphylococcus having nitric oxide synthase can produce NO in the processed meat product to form red nitrosylmyoglobin so that the processed meat product exhibits a red color; and meanwhile, the addition of L-arginine can further improve the red color. However, due to low expression of nitric oxide synthase in coagulase-negative Staphylococcus, its color forming effect is generally lower than that of nitrite. The patent application CN114568644A discloses a processing method for promoting improvement of a red color in fermented sausage. In this method, the fermented sausage inoculated with coagulase-negative Staphylococcus is treated through a low-strength high-hydrostatic pressure, and by fermentation, the a value of the sausage finished product reaches 6.5-7.0. When in use, the inoculation amount of coagulase-negative Staphylococcus in sausage meat is 10⁶ CFU/g-10⁷ CFU/g meat; the treatment pressure of the high hydrostatic pressure is 200 MPa-300 MPa, and the treatment time is 3 min-7 min. By improving the expression amount of nitric oxide synthase in coagulase-negative Staphylococcus and then promoting the yield of nitrosylmyoglobin in fermented sausage, the low-strength high hydrostatic pressure promotes the color of the fermented sausage. This processing method can promote the color of the fermented sausage, thereby providing a feasible solution for coloring replacement of nitrite in the fermented sausage and meeting people's demands on healthy and safe fermented sausage products. However, since the high hydrostatic pressure can cause a reduction in the volume of food, this technology is only suitable for soft packaged foods. Furthermore, the high hydrostatic pressure technology cannot handle a large amount of raw materials once, resulting in high costs. Therefore, in commercial products, ultra-high pressure is often used to process high-end products, such as seafood and rare fruits. The patent application CN115232830A discloses a meat color former based on recombinant bacterial nitric oxide synthase. According to this method, a large amount of bacterial nitric oxide synthase through recombinant expression. When in use, the inoculation amount of the coagulase-negative Staphylococcus suspension is 6.0 log CFU/g-7.5 log CFU/g meat; the addition amount of the recombinant bacterial crude extract is 3.8 mg/100 g-4.2 mg/100 g meat. The recombinant bacterial nitric oxide synthase has high enzyme activity, can catalytically produce a large amount of NO, but when only use in fermented sausage, the recombinant bacterial nitric oxide synthase cannot produce sufficient amounts of NO due to the lack of enough reductase and various cofactors. To solve the lack of cofactors and reductase, Staphylococcus vitulinus is inoculated in the process of making sausage, that is, substances such as cofactors required for catalytic reaction of nitric oxide synthase is provided by adding bacteria. Compared with pure recombinant bacterial nitric oxide synthase, the simultaneous inoculation of Staphylococcus vitulinus can produce a large amount of NO, and is effectively combined with myoglobin in meat products to produce nitrosylmyoglobin, thereby achieving a good color forming effect. When the recombinant enzyme in the application CN115232830A is used alone, it has an insufficient ability of producing NO due to the lack of necessary cofactors and reductase, leading to the content of nitrosylmyoglobin in meat products being far less than that in meat products with nitrite.

Although the above recombinant bacterial nitric oxide synthase has an effect of promoting the formation of the color of meat products, due to the insufficient ability of producing NO in meat products, the content of nitrosylmyoglobin in meat products is far less than that in meat products with nitrite, and the color forming effect of the meat is far weaker than the color forming effect of meat with nitrite. Furthermore, the inoculation of a large amount of Staphylococcus vitulinus can also force changes in the production process of meat products, especially when making non-fermented meat products, it is quite inconvenient. Therefore, it is necessary to seek a new method for obtaining nitric oxide synthase with higher activity and apply it into meat products.

### SUMMARY

The main objective of the present disclosure is to provide a color former based on a fusion enzyme producing nitric oxide and use thereof.

In order to achieve the above objective, the technical solution adopted by the present disclosure is as follows:

The embodiment of the present disclosure provides a color former based on a fusion enzyme producing nitric oxide, wherein the color former comprises a fusion enzyme producing nitric oxide, and the fusion enzyme is formed by sequentially combining a nitric oxide synthase, a flavoprotein and a flavoprotein reductase pairwise via linker peptides.

The embodiment of the present disclosure also provides use of a fusion enzyme producing nitric oxide in preparing a color former, wherein the fusion enzyme is formed by sequentially combining nitric oxide synthase, flavoprotein and flavoprotein reductase pairwise via linker peptides.

The embodiment of the present disclosure also provides use of the above color former based on the fusion enzyme producing nitric oxide in color formation of meat products.

The embodiment of the present disclosure also provides a color forming method of a color former, comprising mixing and incubating the color former with a substrate so as to achieve the color formation of the substrate;
wherein, the color former comprises the above color former based on the fusion enzyme producing nitric oxide; and the substrate comprises metmyoglobin or meat products.

Compared with the prior art, the present disclosure has the beneficial effects: reductase domain YkuN-YumC is directly linked to bacterial nitric oxide synthase through gene fusion, which reduces the complexity of redox chains, improves the efficiency of electron transfer and provides the reductase domain required for bacterial nitric oxide synthase catalysis, thereby significantly improving the NO producing efficiency of the fusion enzyme and effectively combing with myoglobin in meat products to produce nitrosylmyoglobin, so as to achieve the color forming effect equivalent to that of meat products added with sodium nitrite. The fusion enzyme prepared in the present disclosure can effectively replace the color forming effect of nitrite in meat processing, and provides a highly practical solution for nitrite color forming replacement of meat products and improvement of its safety. The present disclosure provides a concept of enzymatic color formation. The use of enzymes alone can directly make meat products colored, retain the original making process and traditional flavor of meat products and reduce the use of nitrite in meat products, thereby facilitating green food production, and promoting food safety.

### BRIEF DESCRIPTION OF THE DRAWINGS

In order to more clearly illustrate the technical solutions in the embodiments of the present disclosure or in the prior art, the drawings required to be used in the embodiments or in the prior art will be simply described, obviously, the drawings described below are only are some embodiments of the present disclosure, other drawings can also be obtained by persons of ordinary skill in the art without creative efforts according to these drawings.

FIG. 1 is a sodium dodecyl sulfate-polyacrylamide gel electrophoresis (SDS-PAGE) analytic graph of a recombinant bacterial crude extract and a recombinant enzyme solution prepared in comparative example 1, and a fusion bacterial crude extract and a fusion enzyme solution prepared in example 1; wherein lane 1 and lane 2 respectively correspond to the recombinant bacterial crude extract and the recombinant enzyme solution prepared in comparative example 1, and lane 3 and lane 4 respectively correspond to the fusion bacterial crude extract and the fusion enzyme solution prepared in example 1;

FIG. 2A-FIG. 2B are hemoglobin absorption spectrum curve graphs of different treatment groups added with comparative example 1 or example 1 in test example 4 of the present disclosure; and

FIG. 3A-FIG. 3B are hemoglobin absorption spectrum curve graphs of minced meat in different treatment groups in example 2 and comparative examples 2-4 of the present disclosure.

### DETAILED DESCRIPTION OF THE EMBODIMENTS

In view of the defects in the prior art, the inventor of this case proposes the technical solution of the present disclosure through long-term research and extensive practice. The following description will provide a clear and complete description of the technical solution of the present disclosure. Obviously, the described embodiments are some embodiments of the present disclosure, but not all the embodiments. Based on the embodiments of the present disclosure, other embodiments obtained by persons of ordinary skill in the art without creative efforts are all included within the scope of protection of the present disclosure.

Specifically, as an aspect of the technical solution of the present disclosure, a color former based on a fusion enzyme producing nitric oxide comprises the fusion enzyme producing nitric oxide; the fusion enzyme is formed by sequentially combining nitric oxide synthase, flavoprotein and flavoprotein reductase pairwise via linker peptides.

Further, the amino acid sequence of the fusion enzyme is formed by combining nitric oxide synthase, flavoprotein and flavoprotein reductase with two linker peptides. Specifically, the nitric oxide synthase, flavoprotein and flavoprotein reductase are sequentially combined pairwise via the linker peptides to form a fusion enzyme.

When the recombinant enzyme in the application CN115232830A is used alone, it has an insufficient ability of producing NO due to the lack of necessary cofactors and reductase, leading to the content of nitrosylmyoglobin in meat products being far less than that in meat products added with nitrite. YkuN and YumC are flavoprotein and flavoprotein reductase derived from Bacillus subtilis. The flavoprotein is an electron carrier, which can provide electrons for various enzymes including p450. The inventor finds that YkuN-YumC is directly linked to the nitric oxide synthase through gene fusion, so that the enzyme activity of the nitric oxide synthase is enhanced to significantly improve the color forming effect.

In some preferred embodiments, a method for preparing the fusion enzyme comprises:
providing a fusion plasmid at least containing a nitrogen oxide synthase gene, a flavoprotein gene and a flavoprotein reductase gene;
transforming B. subtilis with the fusion plasmid to obtain a fusion strain;
and culturing the fusion strain, mixing and incubating the fusion strain subjected to wall breaking with a Ni-NTA agarose purification resin, and then isolating to obtain the fusion enzyme.

Further, the method for preparing the fusion enzyme specifically comprises:
a1, in a polymerase chain reaction (PCR) system, amplifying nitric oxide synthase gene, flavoprotein gene and flavoprotein reductase gene fragments based on B. subtilis 168 genomic DNA as a template; wherein the upstream primer of the nitric oxide synthase used in the PCR system has a sequence as shown in SEQ ID NO: 1, and the downstream primer of the nitric oxide synthase has a sequence as shown in SEQ ID NO: 2 or SEQ ID NO: 3; the upstream primer of the used flavoprotein has a sequence as shown in SEQ ID NO: 4, and the downstream primer of the flavoprotein has a sequence as shown in SEQ ID NO: 5; the upstream primer of the used flavoprotein reductase has a sequence as shown in SEQ ID NO: 6, and the downstream primer of the flavoprotein reductase has a sequence as shown in SEQ ID NO: 7;
a2, in the PCR system, amplifying a linearized plasmid based on pP43NMK as a template to obtain a linearized pP43NMK plasmid; wherein the upstream primer of the pP43NMK used in the PCR system has a sequence as shown in SEQ ID NO: 8, and the downstream primer of the pP43NMK has a sequence as shown in SEQ ID NO: 9;
a3, adding a FastDigest DpnI enzyme into the linearized pP43NMK plasmid obtained in step a2, and treating the above plasmid on a PCR instrument to eliminate a cyclic plasmid, and then inactivating the FastDigest DpnI enzyme to obtain a pP43NMK linear plasmid;
a4, in a PCR1 system, fusing the nitric oxide synthase gene, flavoprotein gene and flavoprotein reductase gene fragments obtained in step a1 through triple fusion PCR to obtain a fusion fragment, and then further amplifying the fusion fragment in a PCR2 system to obtain a large amount of fusion fragment amplification products;
a5, seamlessly linking the fusion fragment amplification product obtained in step a4 with the pP43NMK linear plasmid obtained in step a3 to obtain fusion plasmid A; then mixing and incubating the fusion plasmid A with Escherichia coli JM109 competent cells to obtain E. coli JM109 containing the fusion plasmid A;
a6, culturing the E. coli JM109 containing the fusion plasmid A in an LB culture medium containing ampicillin to obtain thalli, and then performing plasmid extraction on the thalli to obtain fusion plasmid B;
a7, adding a histidine label at the terminal of the flavoprotein reductase gene in the fusion plasmid B obtained in step a6 to obtain E. coli TOP10 containing the fusion plasmid; and
a8, culturing the E. coli TOP10 containing the fusion plasmid obtained in step a7 in an LB culture medium containing ampicillin to obtain thalli, and then performing plasmid extraction on the thalli to obtain the fusion plasmid.

Further, the method for preparing the fusion enzyme specifically comprises: thawing B. subtilis 168 competent cells at a room temperature, and adding the obtained fusion plasmids into the thawed cells for incubation, culture and screening to obtain the fusion strain.

Further, the method for preparing the fusion enzyme specifically comprises:
c1, inoculating the single colony in the obtained fusion strain to a culture medium containing kanamycin to be cultured, and then isolating the strain from the culture system;
c2, sufficiently washing the strain isolated in step c1 and then performing wall breaking treatment on the strain, and then isolating to obtain supernatant, so as to obtain a fusion bacterial crude extract; and
c3, mixing and incubating the fusion bacterial crude extract obtained in step c2 with the Ni-NTA agarose purification resin, and performing protein elution to obtain a fusion enzyme solution; preferably, the concentration of the fusion enzyme in the fusion enzyme solution is 0.1 mg/L-0.5 mg/L.

In some preferred specific embodiments, the method for preparing the fusion enzyme mainly comprises:
Y1, providing a fusion plasmid containing nitric oxide synthase gene, flavoprotein gene and flavoprotein reductase gene fragments;
Y2, transforming B. subtilis with the fusion plasmid obtained in step Y1 to obtain a fusion strain; and
Y3, culturing the fusion strain obtained in step Y2, performing wall breaking on the cultured fusion strain and then mixing and incubating the fusion strain subjected to wall breaking with a Ni-NTA agarose purification resin, and then isolating to obtain the fusion enzyme.

In one embodiment, the method for preparing the fusion plasmid obtained in step Y1 specifically comprises the following steps:
a1, in a PCR system, amplifying nitric oxide synthase gene, flavoprotein gene and flavoprotein reductase gene fragments based on B. subtilis 168 genomic DNA as a template;
a2, in the PCR system, amplifying a linearized plasmid based on pP43NMK as a template to obtain a PCR product, namely, a linearized pP43NMK plasmid;
a3, adding a FastDigest DpnI enzyme into the linearized pP43NMK plasmid PCR product obtained in step a2, and treating the above plasmid for 10 min at 37° C. on a PCR instrument to eliminate a cyclic plasmid, and then inactivating the FastDigest DpnI enzyme for 10 min at 85° C. to obtain a pP43NMK linear plasmid;
a4, in a PCR1 system, fusing the nitric oxide synthase gene, flavoprotein gene and flavoprotein reductase gene fragments obtained in step a1 through triple fusion PCR to obtain a fusion fragment, and then further amplifying the fusion fragment in a PCR2 system to obtain a large amount of fusion fragment amplification products;
a5, seamlessly linking the fusion fragment amplification product obtained in step a4 with the pP43NMK linear plasmid obtained in step a3 to obtain fusion plasmid A; adding 10 μL of fusion plasmid A into 100 μL of E. coli JM109 competent cells to undergo an ice bath for 30 min and subsequently performing heat shock on the cells for 45 s in a water bath at 42° C., and then instantly placing the cells subjected to heat shock on ice for 3 min; adding 1 mL of LB culture medium, performing oscillatory incubation for 1 h at 37° C., subsequently centrifuging to discard 900 μL of supernatant, resuspending the remaining culture with the remaining culture medium and coating the resuspended culture onto an LB plate containing ampicillin to be cultured for 16 h at 37° C., so as to obtain E. coli JM109 containing the fusion plasmid A;
a6, culturing the E. coli JM109 containing the fusion plasmid A obtained in step a5 in an LB culture medium containing ampicillin for 12 h, centrifuging a culture solution for 2 min at 8000 g at 0° C.-4° C. to obtain thalli, and then extracting plasmids with DiaSpin column plasmid DNA small extraction kit to obtain fusion plasmid B;
a7, adding a histidine label at the terminal of the flavoprotein reductase gene in the fusion plasmid B obtained in step a6 to obtain E. coli TOP10 containing the fusion plasmid; and
a8, culturing the E. coli TOP10 containing the fusion plasmid in an LB culture medium containing ampicillin for 12 h, centrifuging a culture solution for 2 min at 8000 g at 0° C.-4° C. to obtain thalli, and then extracting plasmids with DiaSpin column plasmid DNA small extraction kit to obtain the fusion plasmid.

Further, the PCR system in step a1 includes: 2 μL of each of 10 mM upstream and downstream primers, 25 μL of PrimeSTAR Max DNA Polymerase, 21 μL of sterile water and 0.5 μL of 25 mmol/L MgCl₂, and a few of B. subtilis 168 single colonies are picked with an inoculating loop and accessed to this system and uniformly stirred; the PCR reaction program is as follows: pre-denaturation for 15 min at 95° C., 30 s at 95° C., 30 s at 56° C. and 2 min at 72° C., which is one cycle with 35 cycles in total, and finally extension is continued for 5 min.

Further, the PCR system in step a2 includes: 2 μL of each of 10 mM upstream and downstream primers, 2 μL of 20 ng/μL-100 ng/μL pP43NMK plasmid, 25 μL of PrimeSTAR Max DNA Polymerase, and 19 μL of sterile water; the PCR reaction program is as follows: pre-denaturation for 5 min at 95° C., 30 s at 95° C., 30 s at 56° C. and 2 min at 72° C., which is one cycle with 35 cycles in total, and finally extension is continued for 5 min.

Further, the PCR1 system in step a4 includes: 10 μL of PrimeSTAR Max DNA Polymerase, 10 μL of nitric oxide synthase sequence, flavoprotein sequence and flavoprotein reductase sequence (a concentration ratio is 1:2:1); the PCR1 reaction program is as follows: pre-denaturation for 3 min at 95° C.; 30 s at 95° C., 30 s at 56.8° C. and 2 min at 72° C., which is one cycle with 15 cycles in total; and finally extension is continued for 5 min; the PCR2 system includes: 2 μL of fusion fragment, 2 μL of each of 10 mM upstream and downstream primers, 25 μL of PrimeSTAR Max DNA Polymerase, and 19 μL of sterile water; the PCR2 reaction program is as follows: pre-denaturation for 3 min at 95° C.; 30 s at 95° C., 30 s at 56° C. and 2 min at 72° C., which is one cycle with 35 cycles in total; and finally extension is continued for 5 min.

In one embodiment, the preparation method of the fusion strain in step Y2 specifically comprises the following steps: thawing the prepared B. subtilis 168 competent cells at a room temperature, adding the fusion plasmid in step Y1, performing oscillatory incubation for 2 h at 37° C. at 200 rpm, and subsequently coating the incubated cells onto an LB plate containing kanamycin to be cultured for 18 h to obtain the fusion strain.

In one embodiment, the fusion enzyme in step Y3 is a purified fusion enzyme solution whose preparation method comprises the following steps:
c1, picking the single colony of the fusion strain obtained in step Y2 to be inoculated to a culture medium containing kanamycin for culture, and then isolating the strain in the culture system;
c2, sufficiently washing the strain isolated in step c1 and then performing wall breaking treatment on the strain, and then isolating to obtain supernatant, so as to obtain a fusion bacterial crude extract; and
c3, mixing and incubating the fusion bacterial crude extract obtained in step c2 with the Ni-NTA agarose purification resin, and then performing protein elution to obtain the purified fusion enzyme solution.

Further, the preparation method of the fusion enzyme solution comprises the following steps:
c1, picking the single colony of the fusion strain to be inoculated to a TB culture medium containing kanamycin, performing shaking culture for 18 h-20 h at 36° C.-38° C., and then taking a precipitate to obtain thalli;
c2, washing the thalli obtained in step c1 with 15 mM-20 mM phosphate buffer saline (PBS) with a pH value of 7.4-7.5 for 3-5 times, performing ultrasonic wall breaking on the thalli for 12 min-15 min at 0° C.-4° C. and then centrifuging the thalli for 5 min-8 min at 0° C.-4° C. at the rotation speed of 10000 rpm-12000 rpm, and subsequently taking supernatant, so as to obtain the fusion bacterial crude extract; and
c3, incubating the fusion bacterial crude extract obtained in step c2 with a Ni-NTA agarose purification resin for 1.8 h-2.2 h at 0° C.-4° C., followed by eluting proteins with 750 μL of Elution Buffer with a pH value of 7.8-8.2 for 8-12 times, so as to obtain the fusion enzyme solution, wherein the concentration of the target enzyme solution is 0.1 mg/mL-0.5 mg/mL.

In one embodiment, the wall breaking treatment in step c2 is ultrasonic wall breaking which has the following specific conditions: operating for 2 s-3 s in the frequency of 20 kHz-25 kHz under the power of 100 W-500 W at an interval of 2 s-3 s.

In the present disclosure, a large amount of fusion enzymes are expressed in a food-grade B. subtilis expression system by using fusion and recombinant expression techniques and expression products are added into meat products, which can effectively promote the generation of nitrosylmyoglobin to better improve the color forming effect of the meat products.

Another aspect of the embodiment of the present disclosure also provides use of a fusion enzyme producing nitric oxide in preparing a color former, wherein the fusion enzyme is formed by sequentially combining nitric oxide synthase, flavoprotein and flavoprotein reductase pairwise via linker peptides.

The fusion enzyme provided in the present disclosure has relatively high enzyme activity, can catalytically produce a large amount of nitric oxide and can be effectively combined with the myoglobin in meat products to produce nitrosylmyoglobin, thereby effectively promoting the red color of the meat products so as to obtain the color forming effect equivalent to that of sodium nitrite, and providing a highly practical solution for nitrite color forming replacement of meat products and improvement of its safety.

Another aspect of the embodiment of the present disclosure also provides use of the above color former based on the fusion enzyme producing nitric oxide in color formation of meat products.

Further, the meat products comprise fermented and/or non-fermented meat products.

Further, the meat products comprise sausage, ham, luncheon meat, bacon, jerky and the like.

Another aspect of the embodiment of the present disclosure also provides a color forming method of the color former, comprising: mixing and incubating the color former with a substrate so as to achieve the color formation of the substrate;
wherein, the color former comprises the above color former based on the fusion enzyme producing nitric oxide; and the substrate comprises metmyoglobin or meat products.

In some preferred embodiments, NO catalytically produced by the color former can transform brown metmyoglobin into bright red nitrosylmyoglobin.

In some preferred embodiments, the color forming method specifically comprises: mixing the color former with a substrate and incubating the obtained mixture for 0.5 h-30 h at 4° C.-42° C.

In some preferred embodiments, the concentration of the fusion enzyme in the color former is 0.1 mg/mL-0.5 mg/mL.

In some preferred embodiments, the color forming method specifically comprises: adding the color former into an LB culture medium containing 4 mg/mL-6 mg/mL metmyoglobin and 8 mmol/L-12 mmol/L L-arginine, instantly covering the top of the culture medium with sterile paraffin oil and performing anaerobic incubation for 0.5 h-16 h at 4° C.-42° C.; wherein a volume ratio of the color former to the LB culture medium to the sterile paraffin oil is 100 μL-200 μL: 1 mL-5 mL: 100 μL-300 μL.

In some preferred embodiments, the color forming method specifically comprises: evenly mixing the color former with meat products and accessories to form mixed minced meat and palletizing the mixed minced meat at 15° C.-42° C., covering the palletized minced meat with a plastic wrap to be placed for 6 h-30 h, then storing and processing the obtained mixed minced meat; wherein the accessories comprise L-arginine, sodium chloride and glucose.

Further, a mass ratio of the color former to the meat products is 8 mg-12 mg: 100 g.

Further, a mass ratio of the meat products to L-arginine to sodium chloride to glucose is 100:0.5-1.0:0.5-5.0:0.5-10.

In one embodiment, the color forming method specifically comprises the following steps:
(1) pig hind leg meat is taken to remove fats and connective tissues at 0° C.-4° C., then cut into small pieces and shredded in a meat mincer with a diameter of 1 mm-5 mm to obtain minced meat;
(2) accessories are added into the minced meat obtained in step (1) to be stirred and uniformly mixed, and then a fusion enzyme solution is added for uniform stirring, so as to obtain mixed minced meat;
(3) the mixed minced meat obtained in step (2) is stored, or processed under certain conditions to obtain a meat product.

Further, the accessories in step (1) include but are not limited to the following components in parts by weight: 0.5-1.0 part by weight of L-arginine, 0.5-5.0 parts by weight of sodium chloride and 0.5-10 parts by weight of glucose, based on 100 parts by weight of meat.

Further, the storage method in step (3) specifically comprises: the mixed minced meat is palletized at 15° C.-42° C., then covered with a plastic wrap and placed for 6 h-30 h.

Next, the technical solution of the present disclosure will be described in detail in combination with several preferred examples and drawings. These examples will be implemented on the premise of the technical solution of the present disclosure and give detailed embodiments and specific operation process, but the scope of protection of the present disclosure is not limited to the following examples

The experimental materials used in the following examples, unless otherwise specified, are all purchased by conventional biochemical reagent companies.

Sources and preparation methods of partial raw materials used in the following examples and comparative examples are as follows:

### (1) Experimental Culture Medium

The LB culture medium is purchased from Qingdao Haibo Biotechnology Co., Ltd, and used for activation and passage culture of E. coli and B. subtilis.

The LB agar culture medium is purchased from Qingdao Haibo Biotechnology Co., Ltd, and used for resistance selection and counting of E. coli and B. subtilis.

The TB culture medium is used for amplification culture of B. subtilis. The formula is as follows: 10.0 g of glycerol, 24.0 g of yeast powder, 12.0 g of tryptone, 16.4 g of K₂HPO₄·3H₂O and 2.3 g of KH₂PO₄ in per L of water, and final pH 7.4±0.2.

The GM culture medium mother liquor: 10×minimum salt solution, 50% glucose, 5% hydrolyzed casein, 10% yeast juice, and a calcium and magnesium ion solution. Where, other solution components are sterilized for 15 min by high-pressure steam at 121° C. for later use, except that hydrolyzed casein needs to be sterilized through a sterile filtration membrane in a sterile environment. 100 mL of 10×minimum salt solution: 18.34 g of K₂HPO₄·H₂O, 6.0 g of KH₂PO₄, 2.0 g of (NH₄)₂SO₄, 1.0 g of trisodium citrate dihydrate and 0.2 g of MgSO₄·7H₂O are dissolved with distilled water, and diluted to 100 mL. 20 mL of calcium and magnesium ion solution: 2.03 g of MgCl₂·6H₂O and 0.22 g of CaCl₂ are dissolved with distilled water, and diluted to 20 mL.

5 mL of GMI culture medium: 500 μL of 10×minimum salt solution, 50 μL of 50% glucose, 20 μL of 5% hydrolyzed casein, 50 μL of 10% yeast juice and 4.38 mL of sterile water.

90 mL of GM II culture medium: 8.8 mL of 10×minimum salt solution, 900 μL of 50% glucose, 72 μL of 5% hydrolyzed casein, 36 μL of 10% yeast juice, 450 μL of calcium and magnesium ion solution and 79.742 mL of sterile water.

Ampicillin and kanamycin are selectively added into the culture medium. Ampicillin is added when the transformed E. coli is cultured, kanamycin is added when the transformed B. subtilis is cultured, and the working concentration is 100 μg/mL.

The Competent Cell Preparation Kit is purchased from Baori Medical Biotechnology (Beijing) Co., Ltd, and used for construction of plasmids.

The ClonExpress II One Step Cloning Kit is purchased from Nanjing Novozan Biotechnology Co., Ltd., and used for construction of plasmids.

The DiaSpin Column Plasmid DNA Small Extraction Kit is purchased from Shanghai Shenggong Biotechnology Co., Ltd, and used for extraction of plasmids.

The SDS-PAGE gel preparation kit is purchased from Beijing Solarbio Biotechnology Co., Ltd, and used for preparing gel.

The Bradford protein concentration determination kit is purchased from Shanghai Beyoyime Biotechnology Co., Ltd, and used for determination of protein concentration.

Addition of histidine label in plasmids: Qingke Biotechnology Co., Ltd. is entrusted to add a histidine label to the terminal of the flavoprotein reductase gene in fusion plasmid B to obtain a fusion plasmid; Qingke Biotechnology Co., Ltd. is entrusted to add the histidine label to the terminal of the nitric oxide synthase gene of the recombinant plasmid B to obtain a recombinant plasmid.

(2) Preparation of E. coli JM109 Competent Cells

E. coli JM109 preserved in a glycerinum tube is picked and inoculated into 10 mL of LB liquid culture medium, and then subjected to shaking culture for 12 h at 37° C. at the rotation speed of 200 rpm to activate strains. According to the specification, the Competent Cell Preparation kit is used to prepare E. coli competent cells. The prepared E. coli competent cells are stored at −80° C. for later use.

(3) Preparation of B. subtilis 168 Competent Cells

The strains of B. subtilis 168 preserved on an inclined plane are picked into 5 mL of GM I culture medium to be activated, and then subjected to shaking culture for 13 h at 37° C. at 200 rpm. 1 mL of culture solution is added into 9 mL of GM I culture medium, and continued to be cultured for 3.5 h under the same conditions. 10 mL of the above culture solution is taken and inoculated into 90 mL of GM II culture solution, and then cultured for 1.5 h under the same conditions. 10 mL of the above GM II culture solution is taken and subpackaged into 50 mL of sterile centrifuge tube, and then centrifuged for 5 min under the conditions of 4° C. and 3000 rpm. 9 mL of supernatant is removed, 1 mL of the above supernatant is left to resuspend thalli, and then the resuspended thalli are subpackaged into 1.5 mL of centrifuge tube, with 500 μL of thalli and 250 μL of 30% glycerin in each tube. Finally, the thalli are cryopreserved at −80° C. for later use.

### (4) Primers Used in Examples are Shown in Table 1 Below.

TABLE 1
Primers used in examples of the present disclosure

| Primer name | Primer sequence (5′ to 3′) |
|---|---|
| nos-F | GAGAGGAATGTACACATGGAAGAAAAAGAAATACTCTGGAACG |
| (SEQ ID NO: 1) | |
| nos-R1 | TACGCCAAGCTTTCATTACTCATAAGGCTTATCTTGATAAAAATAGTTC |
| (SEQ ID NO: 2) | |
| pP43NMK-F | TGAAAGCTTGGCGTAATCATG |
| (SEQ ID NO: 8) | |
| pP43NMK-R | GTGTACATTCCTCTCTTACCTATAATG |
| (SEQ ID NO: 9) | |
| nos-R2 | GGTGGTGGTGGTAATGGTGGTGGTGGTTCTGGTGGTGGTGGTTCCTCATA |
| (SEQ ID NO: 3) | AGGCTTATCTTGATAAAAATAG |
| YkuN-F | ATTACCACCACCACCATTACCACCACCACCATTACCTCCTCCACCAATGGC |
| (SEQ ID NO: 4) | TAAAGCCTTGATTACATATG |
| YkuN-R | GAGGAGGAGGAGGAAGAGGAGGAGGAGGCTCAGGAGGAGGAGGCTCTG |
| (SEQ ID NO: 5) | AAACATGGATTTTTTCCTTGTTCATATAATC |
| YumC-F | TTCCTCCTCCTCCTCTTCCTCCTCCTCCTCTTCCACCACCACCAATGCGAG |
| (SEQ ID NO: 6) | AGGATACAAAGGTTTATGATATTAC |
| YumC-R | TACGCCAAGCTTTCATTATTTATTTTCAAAAAGACTTGTTGAGTG |
| (SEQ ID NO: 7) | |

(5) Experimental strains: E. coli JM109 with an article number of C1300, Beijing Zhuangmeng International Biogene Technology Co., Ltd. B. subtilis 168, ATCC23857, Bio Vector NTCC Typical Culture Collection Center.

(6) Preparation method of metmyoglobin: a 50 mg/mL myoglobin stock solution stored in 50 mM PBS (pH 6.0) is heated for 30 min at 50° C. to inactivate the metmyoglobin reductase, a denatured protein is removed by centrifugation (10,000 g, 5 min, 4° C.), the obtained supernatant contains about 80% or more of metmyoglobin, and filtration sterilization is performed on the metmyoglobin solution via a 0.22 μm filter membrane.

### Example 1

A color former based on a fusion enzyme producing nitric oxide mainly comprised the fusion enzyme producing nitric oxide, wherein a method for preparing the fusion enzyme comprised the following steps:
Y1, a fusion plasmid containing nitric oxide synthase gene, flavoprotein gene and flavoprotein reductase gene fragments was provided, and a method for preparing the fusion plasmid specifically comprised the following steps:
a1, in 50 μL of PCR system, nitric oxide synthase gene, flavoprotein gene and flavoprotein reductase gene fragments were amplified based on B. subtilis 168 genomic DNA as a template; the PCR system was as follows: 2 μL of each of 10 mM upstream and downstream primers, 25 μL of PrimeSTAR Max DNA Polymerase, 21 μL of sterile water and 0.5 μL of 25 mmol/L MgCl₂, and a few of B. subtilis 168 single colonies were picked with an inoculating loop and accessed to this system and uniformly stirred; the PCR reaction program was as follows: pre-denaturation for 15 min at 95° C., 30 s at 95° C., 30 s at 56° C. and 2 min at 72° C., which was one cycle with 35 cycles in total, and finally extension was continued to be conducted for 5 min;
a2, in 50 μL of PCR system, a linearized plasmid was amplified based on pP43NMK as a template to obtain a PCR product, i.e., a linearized pP43NMK plasmid; the PCR system was as follows: 2 μL of each of 10 mM upstream and downstream primers, 2 μL of 50 ng/μL pP43NMK plasmid, 25 μL of PrimeSTAR Max DNA Polymerase, and 19 μL of sterile water; the PCR reaction program was as follows: pre-denaturation for 5 min at 95° C., 30 s at 95° C., 30 s at 56° C. and 2 min at 72° C., which was one cycle with 35 cycles in total, and finally extension was continued to be conducted for 5 min;
a3, 1 μL of FastDigest DpnI enzyme was added into 50 μL of linearized pP43NMK plasmid PCR product obtained in step a2, and the above plasmid was treated for 10 min at 37° C. on a PCR instrument to eliminate a cyclic plasmid, and then the FastDigest DpnI enzyme was inactivated for 10 min at 85° C. to obtain a pP43NMK linear plasmid;
a4, in 20 μL of PCR1 system, the nitric oxide synthase gene, flavoprotein gene and flavoprotein reductase gene fragments obtained in step a1 were fused through triple fusion PCR to obtain a fusion fragment; the PCR1 system was as follows: 10 μL of PrimeSTAR Max DNA Polymerase, and 10 μL of nitric oxide synthase sequence, flavoprotein sequence and flavoprotein reductase sequence (a concentration ratio is 1:2:1); the PCR1 reaction program was as follows: pre-denaturation for 3 min at 95° C.; 30 s at 95° C., 30 s at 56.8° C. and 2 min at 72° C., which was one cycle with 15 cycles in total; and finally extension was continued for 5 min; the fusion fragment was taken and further amplified, and 50 μL of PCR2 system in total, which included: 2 μL of fusion fragment, 2 μL of each of 10 mM upstream and downstream primers, 25 μL of PrimeSTAR Max DNA Polymerase, and 19 μL of sterile water; the PCR2 reaction program was as follows: pre-denaturation for 3 min at 95° C.; 30 s at 95° C., 30 s at 56° C. and 2 min at 72° C., which was one cycle with 35 cycles in total; and finally extension was continued to be conducted for 5 min; a large amount of fusion fragment amplification products were obtained;
a5, the fusion fragment amplification product obtained in step a4 was seamlessly linked with the pP43NMK linear plasmid obtained in step a3 by using ClonExpress II One Step Cloning Kit to obtain fusion plasmid A; 10 μL of fusion plasmid A was added into 100 μL of E. coli JM109 competent cells to undergo an ice bath for 30 min and subsequently subjected to heat shock for 45 s in a water bath at 42° C., then the product subjected to heat shock was instantly placed on ice for 3 min; 900 μL of LB culture medium was added, oscillatory incubation was performed for 1 h at 37° C., subsequently the culture solution was centrifuged and 900 μL of supernatant was discarded, the remaining culture was resuspended with the remaining culture medium and coated onto an LB plate containing ampicillin, and then cultured for 16 h at 37° C. to obtain E. coli JM109 containing the fusion plasmid A;
a6, the E. coli JM109 containing the fusion plasmid A was cultured in an LB culture medium containing ampicillin for 12 h, a culture solution was centrifuged for 2 min at 8000 g at 0° C.-4° C. to obtain thalli, and then plasmid extraction was performed on the obtained thalli using DiaSpin column plasmid DNA small extraction kit to obtain fusion plasmid B;
a7, a histidine label was added at the terminal of the flavoprotein reductase gene in the fusion plasmid B obtained in step a6 to obtain E. coli TOP10 containing the fusion plasmid; the sequence of the histidine label was CATCACCATCACCATCAC, as shown in SEQ ID NO: 10;
a8, the E. coli TOP10 containing the fusion plasmid was cultured in 5 mL of LB culture medium containing ampicillin for 12 h, a culture solution was centrifuged for 2 min at 8000 g at 0° C.-4° C. to obtain thalli, and then plasmid extraction was performed on the obtained thalli using DiaSpin column plasmid DNA small extraction kit to obtain the fusion plasmid;
Y2, B. subtilis was transformed with the fusion plasmid obtained in step Y1 to obtain a fusion strain, which specifically comprised: B. subtilis 168 competent cells were thawed at a room temperature, and the fusion plasmids extracted in step b1 were added for oscillatory incubation for 2 h at 200 rpm at 37° C., and subsequently the incubated cells were coated onto an LB plate containing kanamycin to be cultured for 18 h, so that the B. subtilis 168 was transformed with the fusion plasmid to obtain the fusion strain;
Y3, the fusion strain obtained in step Y2 was cultured, and then isolated to obtain the fusion enzyme, which specifically comprised:
c1, the single colony of the fusion strain obtained in step Y2 was picked to be inoculated to 600 mL of TB culture medium containing kanamycin for 20 h of shaking culture at about 37° C. at the rotation speed of 200 rpm, the culture solution was taken and centrifuged for 3 min at 10000 rpm at 4° C., and then a precipitate was taken to obtain thalli;
c2, the thalli obtained in step c1 were washed three times using 20 mM PBS with a pH value of 7.4, ultrasonic wall breaking was performed on the thalli for 15 min at 0° C. in a frequency of 25 kHz under the power of 300 W, with operating for 2 s at an interval of 3 s, then the thalli subjected to wall breaking were centrifuged for 5 min at the rotation speed of 10000 rpm at 0° C., and then a supernatant was taken so as to obtain the fusion bacterial crude extract;
c3, the fusion bacterial crude extract obtained in step c2 was incubated with a Ni-NTA agarose purification resin for 2 h at 4° C., followed by eluting proteins using 750 μL of Elution Buffer with a pH value of 8 for 10 times, so as to obtain the fusion enzyme solution, wherein the concentration of the target enzyme solution was 0.5 mg/mL.

### Example 2

A color forming method of meat by using a color former based on a fusion enzyme producing nitric oxide comprised the following steps:
(1) pig hind leg meat was taken to remove fats and connective tissues at 4° C., then cut into small pieces and shredded in a meat mincer with a diameter of 3 mm to obtain minced meat;
(2) 0.8 g of L-arginine, 2.5 g of sodium chloride and 5 g of glucose were added into 100 g of minced meat obtained in step (1) to be stirred and uniformly mixed, then the fusion enzyme solution prepared in example 1 was added in an addition amount of 10 mg/100 g meat to be uniformly stirred, so as to obtain mixed minced meat; and
(3) the mixed minced meat obtained in step (2) was palletized at 25° C., covered with a plastic wrap and then placed for 20 h so as to improve the red color of the minced meat.

### Example 3

A color forming method of meat by using a color former based on a fusion enzyme producing nitric oxide comprised the following steps:
(1) pig hind leg meat was taken to remove fats and connective tissues at 4° C., then cut into small pieces and shredded in a meat mincer with a diameter of 3 mm to obtain minced meat;
(2) 0.8 g of L-arginine, 2.5 g of sodium chloride and 5 g of glucose were added into 100 g of minced meat obtained in step (1) to be stirred and uniformly mixed, then the fusion enzyme solution prepared in example 1 was added in an addition amount of 10 mg/100 g meat to be uniformly stirred, so as to obtain mixed minced meat; and
(3) the mixed minced meat obtained in step (2) was palletized at 37° C., covered with a plastic wrap and then placed for 8 h so as to improve the red color of the minced meat.

### Example 4

A method for promoting the color formation of metmyoglobin by using a color former based on a fusion enzyme producing nitric oxide comprised:

100 μL of 0.5 mg/mL color former (fusion enzyme solution) prepared in example 1 was added into 2 mL of LB culture medium containing 5 mg/mL metmyoglobin and 10 mM L-arginine, the top of the culture medium was instantly covered with 300 μL of sterile paraffin oil, and then the above materials were subjected to anaerobic incubation for 8 h at 37° C. so as to promote the color formation of metmyoglobin.

### Example 5

A method for promoting the color formation of metmyoglobin by using a color former based on a fusion enzyme producing nitric oxide comprised:

200 μL of 0.5 mg/mL color former (fusion enzyme solution) prepared in example 1 was added into 2 mL of LB culture medium containing 5 mg/mL metmyoglobin and 10 mM L-arginine, the top of the culture medium was instantly covered with 300 μL of sterile paraffin oil, and then the above materials were subjected to anaerobic incubation for 8 h at 37° C. so as to promote the color formation of metmyoglobin.

### Example 6

A method for promoting the color formation of metmyoglobin by using a color former based on a fusion enzyme producing nitric oxide comprised:

100 μL of 0.5 mg/mL color former (fusion enzyme solution) prepared in example 1 was added into 2 mL of LB culture medium containing 5 mg/mL metmyoglobin and 10 mM L-arginine, the top of the culture medium was instantly covered with 300 μL of sterile paraffin oil, and then the above materials were subjected to anaerobic incubation for 0.5 h at 37° C. so as to promote the color formation of metmyoglobin.

### Example 7

A method for promoting the color formation of metmyoglobin by using a color former based on a fusion enzyme producing nitric oxide comprised:

100 μL of 0.5 mg/mL color former (fusion enzyme solution) prepared in example 1 was added into 2 mL of LB culture medium containing 5 mg/mL metmyoglobin and 10 mM L-arginine, the top of the culture medium was instantly covered with 300 μL of sterile paraffin oil, and then the above materials were subjected to anaerobic incubation for 16 h at 7° C.

### Comparative Example 1

A method for preparing a meat color former based on a recombinant enzyme comprised the following steps:

With reference to the method in example 1 in CN115232830A, nitric oxide synthase recombinant bacteria were prepared and plasmid modification was performed.
Y1, a recombinant plasmid containing a nitric oxide synthase gene was provided, and the preparation method of the recombinant plasmid specifically comprised:
a1, in 50 μL of PCR system, a nitric oxide synthase gene fragment was amplified based on B. subtilis 168 genomic DNA as a template; the PCR system was as follows: 2 μL of each of 10 mM upstream and downstream primers, 25 μL of PrimeSTAR Max DNA Polymerase and 21 μL of sterile water, and a few of B. subtilis 168 single colonies were picked with an inoculating loop and accessed to this system and uniformly stirred; the PCR reaction program was as follows: pre-denaturation for 15 min at 95° C., 30 s at 95° C., 30 s at 56° C. and 2 min at 72° C., which was one cycle with 35 cycles in total, and finally extension was continued to be conducted for 5 min;
a2, in 50 μL of PCR system, a linearized plasmid was amplified based on pP43NMK as a template to obtain a PCR product, i.e., a linearized pP43NMK plasmid; the PCR system was as follows: 2 μL of each of 10 mM upstream and downstream primers, 2 μL of 50 ng/μL pP43NMK plasmid, 25 μL of PrimeSTAR Max DNA Polymerase, and 19 μL of sterile water; the PCR reaction program was as follows: pre-denaturation for 5 min at 95° C., 30 s at 95° C., 30 s at 56° C. and 2 min at 72° C., which was one cycle with 35 cycles in total, and finally extension was continued to be conducted for 5 min;
a3, 1 μL of FastDigest DpnI enzyme was added into 50 μL of linearized pP43NMK plasmid PCR product obtained in step a2, and the above plasmid was treated for 10 min at 37° C. on a PCR instrument to eliminate a cyclic plasmid, and then the FastDigest DpnI enzyme was inactivated for 10 min at 85° C. to obtain a pP43NMK linear plasmid;
a4, the nitric oxide synthase gene fragment obtained in step a1 was seamlessly linked with the pP43NMK linear plasmid obtained in step a3 by using ClonExpress II One Step Cloning Kit to obtain fusion plasmid A; 10 μL of fusion plasmid A was added into 100 μL of E. coli JM109 competent cells to undergo an ice bath for 30 min and then heat shock for 45 s in a water bath at 42° C., and then the product subjected to heat shock was instantly placed on ice for 3 min; 900 μL of LB culture medium was added, the plasmid was subjected to oscillatory incubation for 1 h at 37° C. and then centrifuged to discard 900 μL of supernatant, and the remaining culture was resuspended with the remaining culture medium and coated onto an LB plate containing ampicillin, and then cultured for 16 h at 37° C. to obtain E. coli JM109 containing the fusion plasmid A;
a5, the E. coli JM109 containing the fusion plasmid A obtained in step a4 was cultured in an LB culture medium containing ampicillin for 12 h, the culture solution was centrifuged for 2 min at 8000 g at 0° C.-4° C. to obtain thalli, and then plasmid extraction was performed on the obtained thalli by using DiaSpin column plasmid DNA small extraction kit to obtain recombinant plasmid B;
a6, a histidine label was added at the terminal of the nitric oxide synthase gene in the recombinant plasmid B obtained in step a5 to obtain E. coli TOP10 containing the recombinant plasmid; the sequence of the histidine label was CATCACCATCACCATCAC, as shown in SEQ ID NO: 10;
a7, the E. coli TOP10 containing the recombinant plasmid obtained in step a5 was cultured in 5 mL of LB culture medium containing ampicillin for 12 h, the culture solution was centrifuged for 2 min at 8000 g at 4° C. to obtain thalli, and then plasmid extraction was performed on the obtained thalli by using DiaSpin column plasmid DNA small extraction kit to obtain the recombinant plasmid;
Y2, B. subtilis was transformed with the recombinant plasmid obtained in step Y1 to obtain a recombinant strain, which specifically comprised: B. subtilis 168 competent cells were thawed at room temperature, and the recombinant plasmids extracted in step b1 were added for oscillatory incubation for 2 h at 200 rpm at 37° C., and subsequently the cells subjected to oscillatory incubation was coated onto an LB plate containing kanamycin to be cultured for 18 h, so that the B. subtilis 168 was transformed with the recombinant plasmids to obtain the recombinant strain;
Y3, the recombinant strain obtained in step Y2 was cultured and then isolated to obtain the recombinant enzyme, which specifically comprised:
c1, the single colony of the recombinant strain obtained in step b2 was picked and inoculated to 600 mL of TB culture medium containing kanamycin for 20 h of shaking culture at about 37° C. at 200 rpm, the culture solution was taken and centrifuged for 3 min at the rotation speed of 10000 rpm at 4° C., and then a precipitate was taken to obtain thalli;
c2, the thalli were washed three times using 20 mM PBS with a pH value of 7.4, ultrasonic wall breaking was performed on the thalli for 15 min at 0° C. in a frequency of 25 kHz under the power of 300 W, with operating for 2 s at an interval of 3 s, then the bacterial solution was centrifuged for 5 min at the rotation speed of 10000 rpm at 0° C., and then a supernatant was taken so as to obtain the recombinant bacterial crude extract; and
c3, the recombinant bacterial crude extract obtained in step X3 was incubated with a Ni-NTA agarose purification resin for 2 h at 4° C., followed by eluting proteins 10 times using 750 μL of Elution Buffer with a pH value of 8, so as to obtain the recombinant enzyme solution, wherein the concentration of the target enzyme solution was 0.5 mg/mL.

### Comparative Example 2

A meat color forming method of a meat color former based on a recombinant enzyme comprised the following steps:
(1) pig hind leg meat was taken to remove fats and connective tissues at 4° C., then cut into small pieces and shredded in a meat mincer with a diameter of 3 mm to obtain minced meat;
(2) 0.8 g of L-arginine, 2.5 g of sodium chloride and 5 g of glucose were added into 100 g of minced meat obtained in step (1) to be stirred and uniformly mixed, then a recombinant enzyme solution was added in an addition amount of 5 mg/100 g meat to be uniformly stirred, so as to obtain mixed minced meat; and
(3) the mixed minced meat obtained in step (2) was palletized at 25° C., covered with a plastic wrap and then placed for 20 h.

### Comparative Example 3

A color forming method of meat comprised the following steps:
(1) pig hind leg meat was taken to remove fats and connective tissues at 4° C., then cut into small pieces and shredded in a meat mincer with a diameter of 3 mm to obtain minced meat;
(2) 0.8 g of L-arginine, 2.5 g of sodium chloride and 5 g of glucose were added into 100 g of minced meat obtained in step (1) to be stirred and uniformly mixed, so as to obtain mixed minced meat; and
(3) the minced meat obtained in step (2) was palletized at 25° C., covered with a plastic wrap and then placed for 20 h.

### Comparative Example 4

A color forming method of meat utilizing nitrite comprised the following steps:
(1) pig hind leg meat was taken to remove fats and connective tissues at 4° C., then cut into small pieces and shredded in a meat mincer with a diameter of 3 mm to obtain minced meat;
(2) 9 mg of sodium nitrite, 0.8 g of L-arginine, 2.5 g of sodium chloride and 5 g of glucose were added into 100 g of minced meat obtained in step (1) to be stirred and uniformly mixed, so as to obtain mixed minced meat; and
(3) the minced meat obtained in step (2) was palletized at 25° C., covered with a plastic wrap and then placed for 20 h.

### Comparative Example 5

A method for promoting the color formation of metmyoglobin by using a color former based on a fusion enzyme producing nitric oxide comprised:

100 μL of PBS (20 mM, pH 7.4) was added into 2 mL of LB culture medium containing 5 mg/mL metmyoglobin and 10 mM L-arginine, the top of the culture medium was instantly covered with 300 μL of sterile paraffin oil, and then the above materials were subjected to anaerobic incubation for 8 h at 37° C.

### Comparative Example 6

A method for promoting the color formation of metmyoglobin by using a color former based on a fusion enzyme producing nitric oxide comprised:

100 μL of recombinant enzyme solution prepared in comparative example 1 was added into 2 mL of LB culture medium containing 5 mg/mL metmyoglobin and 10 mM L-arginine, the top of the culture medium was instantly covered with 300 μL of sterile paraffin oil, and then the above materials were subjected to anaerobic incubation for 8 h at 37° C.

The concentration of the recombinant enzyme solution was 0.5 mg/mL.

### Test Example 1

### Identification of Fusion Plasmid a and Recombinant Plasmid A

The E. coli JM109 single colonies having ampicillin resistance and containing fusion plasmid A and the E. coli JM109 single colonies containing recombinant plasmid A prepared in example 1 and comparative example 1 were picked and respectively inoculated into 10 mL of LB liquid culture medium containing ampicillin to be cultured for 12 h, plasmid extraction was performed on the cultured bacterial solutions by using DiaSpin Column Plasmid DNA Small Extraction Kit, and then the plasmids were subjected to gene sequencing.

By detection, the base sequences and positions of the fusion gene fragments and nitric oxide synthase gene fragments prepared in example 1 and comparative example 1 are correct, proving the fusion plasmid A and the recombinant plasmid A are successfully constructed.

### Test Example 2

The SDS-PAGE electrophoresis of the fusion bacterial crude extract and the fusion enzyme solution prepared in example 1 and the recombinant bacterial crude extract and the recombinant enzyme solution prepared in comparative example 1

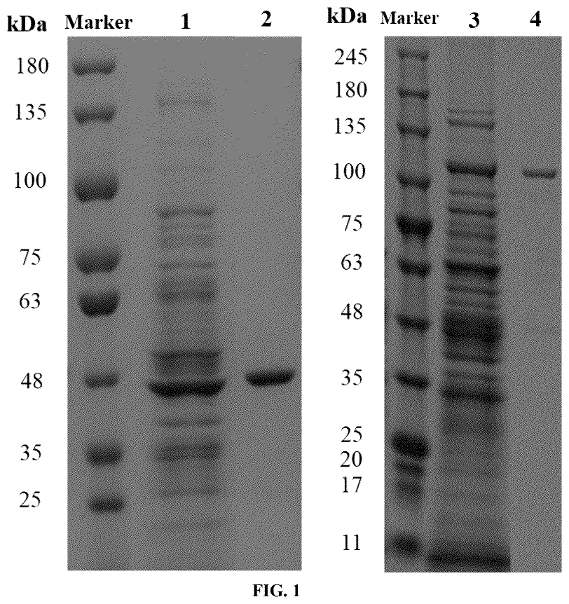

Gel was prepared by using SDS-PAGE gel preparation kit according to the specification, the concentration of the stacking gel was 5%, and the concentration of the separating gel was 10%. The crude extracts prepared in comparative example 1 and example 1 were diluted to 1 mg/mL using 20 mM PBS with a pH value of 7.4, 20 μL of diluted solution was taken and added into 5 μL of 5×protein loading buffer and then boiled for 5 min, and centrifuged for 5 min at 4000 g; 20 μL of supernatant was taken and a protein molecular weight standard was loaded for SDS-PAGE electrophoresis; the electrophoresis conditions were as follows: the voltage was first set to 80 V, the voltage was adjusted to 120 V when electrophoresis reached the separating gel until the end of electrophoresis, and the gel was taken out; the gel was put into a Coomassie brilliant blue staining solution to be dyed for 30 min, and then rinsed several times with a decolorizing solution composed of 100 mL of acetic acid and 900 mL of water until clear strips appear; the test results are shown in FIG. 1, where Lane 1 and Lane 2 correspond to the recombinant bacterial crude extract and recombinant enzyme solution prepared in comparative example 1, respectively, and Lane 3 and Lane 4 correspond to the fusion bacterial crude extract and fusion enzyme solution prepared in example 1, respectively.

By theoretical analysis, the recombinant bacterial crude extract and the fusion bacterial crude extract will have one crude protein at 48 kDa and 101 kDa, respectively, i.e., a recombinant enzyme and a fusion enzyme. It can be seen from FIG. 1 that strips at the above two positions are obviously wider than other strips in the crude extract lane, and therefore it is initially determined that the above two proteins have been successfully and effectively expressed. After the proteins are combined with Ni-NAT agarose purification resin and eluted, it is observed that lane 2 and lane 4 have clear target strips and no scattered strips, proving that the recombinant enzyme and the fusion enzyme are successfully obtained.

### Test Example 3

### Activity Analysis of Bacterial Nitric Oxide Synthase

The enzyme activity analysis was performed on the recombinant enzyme solution prepared in comparative example 1 and the fusion enzyme solution prepared in example 1. The nitric oxide synthase has the function of catalytically producing NO, and the indirect assessment of NOS enzyme activity is performed through the determination of NO oxidation product nitrite. NO, as a free radical having a high reduction degree, is extremely prone to oxidation into nitrite ions, and the nitrite ions were detected through Griess reagent so as to quantitatively analyze NOS enzyme activity. The activity of the enzyme is determined by using a nitric oxide synthase kit.

TABLE 2
Enzyme activity of nitric oxide synthase

| | Comparative example 1 | Example 1 |
|---|---|---|
| Enzyme activity | 1.71 ± 0.26^{b} | 20.20 ± 0.68^{a} |

Note:
different letters represent significant data difference between different groups (P < 0.05).

It can be seen Table 2 that the activities of the nitric oxide synthase and the fusion enzyme determined by the kit are 1.7133±0.2641 U/mgprot and 20.193±0.6809 U/mgprot, respectively. The enzyme activity after fusion expression is about 12 times that of the unmodified enzyme, which indicates that covalently linked flavoprotein and flavoprotein reductase retain the ability of transferring required electrons to the nitric oxide synthase. YkuN and YumC are flavoprotein and flavoprotein reductase which are derived from B. subtilis. The flavoprotein is an electron carrier, which can provide electrons for various enzymes including p450. Flavoprotein YkuN is fused to the N terminal of flavoprotein reductase YumC, because the linking of flavoprotein to the C terminal of YumC may interfere with the function of aromatic residue at the C terminal, the aromatic residue at the C terminal is important for NADPH binding and electron exchange. The internal of the fusion protein is linked by a rigid linker (EPPPP-EPPPP-LPPPP-LPPPP-LPPPP), and a proline-enriched linker can more effectively isolate the domain of the fusion protein. YkuN-YumC is directly linked to nitric oxide synthase through gene fusion, so that the enzyme activity of the nitric oxide synthase is increased to 12 times, indicating that the domain of the supplemented reductase is beneficial for electron transfer. In the present disclosure, the fusion enzyme having extremely high enzyme activity is obtained by utilizing a B. subtilis fusion expression system, so as to provide a feasible solution for the color formation of the fermented sausage.

### Test Example 4

### Promotion of Color Forming Effect of Metmyoglobin

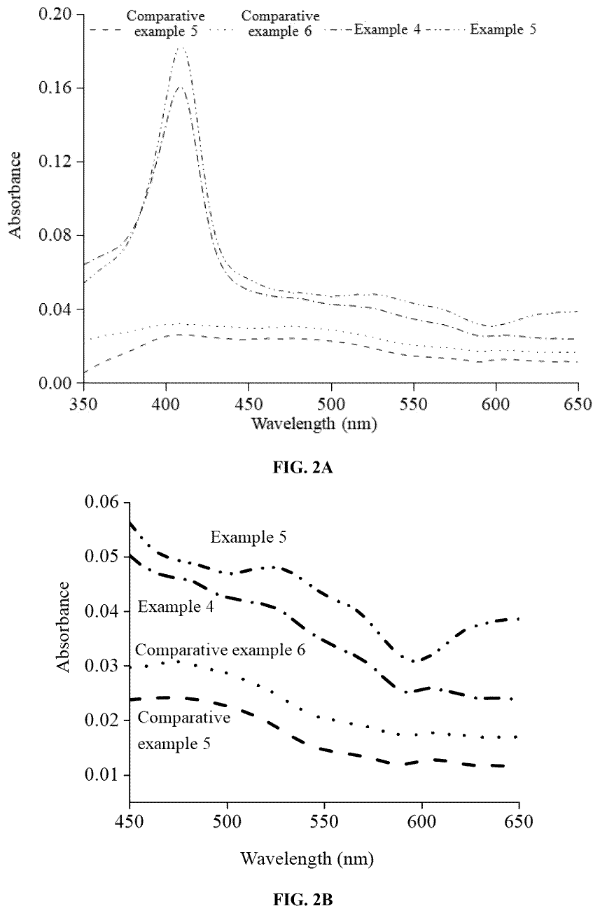

The color of a culture was observed and the a value of each group was measured. The color of the LB culture was measured with a quartz cell (2 mm optical path) under the mode of transmission by using ZE7700 Electric Color Difference Instrument (Nippon Denshoku, Kogyo Co., Tokyo, Japan). The measurement results are seen in Table 3. The culture was centrifuged and precipitated, and the supernatant of the culture medium was used for UV-visible spectrum analysis. The nitrosylmyoglobin was extracted using a 75% (v/v) pre-cooled acetone solution, the supernatant was filtered via a 0.45 μm filter membrane, the filtrate was a NO-Mb extracting solution, and absorption scanning was performed on the filtrate from 350 nm to 650 nm at an interval of 1 nm. The test results are seen in FIG. 2A-FIG. 2B.

TABLE 3
The a value of culture medium in each group

| Group | The a value |
|---|---|
| Comparative example 5 | 3.77 ± 0.01^{d} |
| Comparative example 6 | 4.52 ± 0.02^{c} |
| Example 4 | 5.15 ± 0.02^{b} |
| Example 5 | 5.96 ± 0.04^{a} |

Note:
different letters represent significant data difference between different groups (P < 0.05).

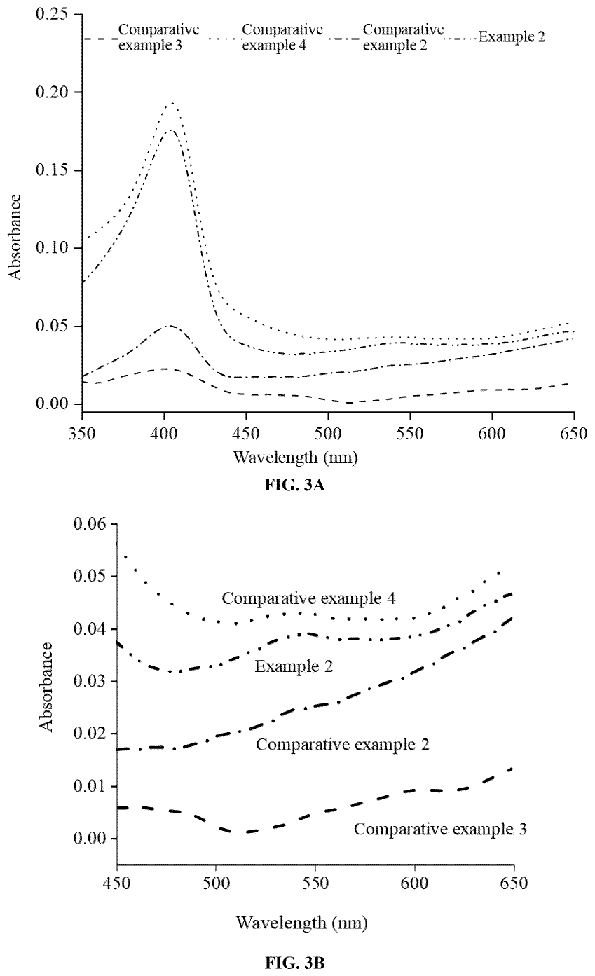

NO produced catalytically by nitric oxide synthase can transform brown metmyoglobin into bright red nitrosylmyoglobin. However, it can be seen from FIGS. 3A-3B and FIGS. 2A-2B that the use of recombinant nitric oxide synthase alone is ineffective, possibly because the reductase and cofactors required for the catalytic action of nitric oxide synthase are lack in the system. After incubation for 8 h at 37° C., the a values of example 4 and example 5 are significantly high (P<0.05), indicating that a large amount of metmyoglobin are transformed into other red myoglobin derivatives in this system. According to UV-visible spectrum scanning results, samples added with fusion enzymes all show maximum peaks, which are typical absorption peaks of nitrosylmyoglobin, at 394 nm and 540 nm. Where, the absorption peak value in example 5 is the highest, indicating that the content of nitrosylmyoglobin in this system is the highest, at this moment, nitrosylmyoglobin has almost not been formed in the systems added with recombinant nitric oxide synthase. It is shown that the fusion enzyme prepared in example 1 has more excellent metmyoglobin transformation ability that the recombinant enzyme solution prepared in comparative example 1.

### Test Example 5

The a values of 4 groups of minced meat prepared in example 2 and comparative examples 2-4 were measured. 5 g of minced meat in each group was taken, the a value of the minced meat was measured using ZE7700 Electric Color Difference Instrument (Nippon Denshoku, Kogyo Co., Tokyo, Japan). The measurement results are seen in Table 4.

TABLE 4
The a values of minced meat prepared in
example 2 and comparative examples 2-4

| Group | The a value of minced meat |
|---|---|
| Comparative example 2 | 4.89 ± 0.12^{b} |
| Comparative example 3 | 4.88 ± 0.19^{b} |
| Comparative example 4 | 7.42 ± 0.46^{a} |
| Example 2 | 6.65 ± 0.35^{a} |

Note:
different letters represent significant survival rate difference between different groups (P < 0.05).

The test results for enzyme activity determination of bacterial nitric oxide synthase indicate that the recombinant enzyme and fusion enzyme expressed and produced in a B. subtilis expression system have intact nitric oxide synthase activity. Therefore, the recombinant enzyme solution and fusion enzyme solution were respectively extracted in comparative example 2 and example 2 and used in minced meat. However, as shown in Table 4, compared with comparative example 3, comparative example 4 and example 2 both have significant promotion effects on the red color formation of minced meat through addition of sodium nitrite and the fusion enzyme, and there is no significant difference between comparative example 4 and example 2 (P>0.05), indicating that the fusion enzyme prepared in example 2 can reach the color forming effect similar to that of nitrite. But, by comparing comparative example 2 with comparative example 3 and example 2, it indicates that the addition of the recombinant enzyme solution difficultly reaches an ideal color forming effect. This is because the structure of the bacterial nitric oxide synthase is different from the structure of the nitric oxide synthase of the mammalian and the bacterial nitric oxide synthase lacks a reductase domain, in bacteria, other reductases are often needed to help the completion of the catalytic reaction of the nitric oxide synthase. Besides, there are also multiple cofactors such as NADPH, FMN, FAD and H₄B/H₄F for assistance. Therefore, in a minced meat system supplemented with only nitric oxide synthase, sufficient amounts of NO cannot be produced due to the lack of sufficient reductases and various cofactors. In example 3 and examples 6-7, different incubation temperatures and time are used respectively, and the color forming effect is similar to that under other incubation conditions and are not described in detail here.

The fusion enzyme solution is added in example 2 of the present disclosure, so as to provide more cofactors and reductases. The a value of the minced meat prepared in example 2 is higher than the a values of fermented sausages prepared in comparative example 2 and comparative example 3, indicating that the fusion enzyme has a considerable promotion effect on the improvement of the a value of the sausage. The a value data indicate that the addition of the fusion enzyme can effectively promote the red color formation of the fermented sausage, and even is equivalent to addition of nitrite, which provides a new and highly practical method for improvement of color forming replacement of nitrite in the fermented sausage and the safety of the fermented sausage.

### Test Example 6

### The Color Forming Effect of Minced Meat

UV-visible spectrum analysis was performed on the systems in example 2 and comparative examples 2-4. 5.0 g of the above minced meat in each group was correctly weighed and added into 45 mL of 75% (v/v) pre-cooled acetone solution, homogenized for 1 min (under the ice bath condition) via a high-speed dispersion machine and centrifuged for 5 min at 4000 g at 4° C., supernatant was taken and filtered via a 0.45 μm filter membrane, and the filtrate was a nitrosylmyoglobin extracting solution. The solution was analyzed by using a UV-visible spectrophotometer, a base line was adjusted using a 75% acetone solution, and then scanning was performed in a wavelength range from 350 nm to 650 nm at an interval of 1 nm. Test results are seen in FIGS. 3A-3B.

The formation of the red color of the minced meat is mainly because nitrosylmyoglobin is present. In order to further analyze possible reasons that the fusion enzyme promotes the improvement of the a value of the minced meat, the nitroso-group pigment of minced meat in each group was extracted and analyzed. By verification of enzyme activity in example 3, the fusion enzyme has higher enzyme activity, and can catalyze the production of a large amount of NO. It can be seen from Table 4 that the individual action of the recombinant enzyme cannot effectively transform metmyoglobin into nitrosylmyoglobin, which is possibly because the meat system lacks reductases and other cofactors required for nitric oxide synthase catalysis. Therefore, the fusion enzyme is added in the minced meat prepared in example 2, and the reduction domain of the reductase is supplemented through fusion expression to expect the supplementation of substances required for nitric oxide synthase catalysis reaction.

It can be seen from results of FIG. 3A-FIG. 3B that except comparative example 3, peak values appear at 394 nm and 540 nm for other groups, which are typical nitrosylmyoglobin absorption peaks, indicating that minced meats prepared in comparative example 2, comparative example 4 and example 2 all have nitrosylmyoglobin. According to absorption peak values, the content of NO-Mb formed in minced meat prepared in comparative example 4 is the maximum, followed by example 2, they are higher than that in minced meats prepared in comparative examples 2-3, which corresponds to the trend of the a value of minced meat in each group. By combining color change in Table 4 and test results of UV-visible analysis, it indicates that compared with the addition of the recombinant enzyme solution in comparative example 2, the addition of the fusion enzyme solution in example 2 can greatly promote the formation efficiency of nitrosylmyoglobin. The fusion enzyme solution added in minced meat prepared in example 2 contains sufficient enzyme activity of nitric oxide synthase and can sufficiently catalyze the reaction of L-arginine to produce a sufficient amount of NO, and NO is combined with myoglobin in minced meat to form a large amount of nitrosylmyoglobin, which is equivalent to the treatment effect of sodium nitrite and provides a practical method for the color forming replacement of nitrite in meat and the improvement of meat safety.

In addition, the inventor of this case conducts tests using other raw materials, process operations and process conditions involved in the specification by reference with the above examples to obtain ideal results.

It should be understood that the technical solution of the present disclosure is not limited to the above specific embodiments, the technical deformations made according to the technical solution of the present disclosure without departing from the purpose of the present disclosure and the scope claimed by claims are all included within the scope of protection of the present disclosure.

What is claimed is:

1. A color former based on a fusion enzyme producing nitric oxide, wherein the color former comprises the fusion enzyme producing the nitric oxide, and the fusion enzyme is formed by sequentially combining a nitric oxide synthase, a flavoprotein, and a flavoprotein reductase pairwise via linker peptides.

2. The color former according to claim 1, wherein a method for preparing the fusion enzyme comprises:
providing a fusion plasmid at least containing a nitrogen oxide synthase gene, a flavoprotein gene, and a flavoprotein reductase gene;
transforming a Bacillus subtilis with the fusion plasmid to obtain a fusion strain; and
culturing the fusion strain, mixing and incubating the fusion strain subjected to wall breaking with a Ni-NTA agarose purification resin, and then isolating to obtain the fusion enzyme.

3. The color former according to claim 2, wherein the method for preparing the fusion enzyme comprises:
a1, in a polymerase chain reaction (PCR) system, amplifying a nitrogen oxide synthase gene fragment, a flavoprotein gene fragment, and a flavoprotein reductase gene fragment based on a B. subtilis 168 genomic DNA as a template; wherein an upstream primer of the nitric oxide synthase used in the PCR system has the sequence as shown in SEQ ID NO: 1, and a downstream primer of the nitric oxide synthase has the sequence as shown in SEQ ID NO: 2 or SEQ ID NO: 3; an upstream primer of the flavoprotein has the sequence as shown in SEQ ID NO: 4, and a downstream primer of the flavoprotein has the sequence as shown in SEQ ID NO: 5; an upstream primer of the flavoprotein reductase has the sequence as shown in SEQ ID NO: 6, and a downstream primer of the flavoprotein reductase has the sequence as shown in SEQ ID NO: 7;
a2, in the PCR system, amplifying a linearized plasmid in the PCR system based on a pP43NMK as a template to obtain a linearized pP43NMK plasmid;
a3, adding a FastDigest DpnI enzyme into the linearized pP43NMK plasmid obtained in step a2, and treating a resulting plasmid on a PCR instrument to eliminate a cyclic plasmid, and then inactivating the FastDigest DpnI enzyme to obtain a pP43NMK linear plasmid;
a4, in a PCR1 system, fusing the nitrogen oxide synthase gene fragment, the flavoprotein gene fragment, and the flavoprotein reductase gene fragment obtained in step a1 through a triple fusion PCR to obtain a fusion fragment, and then further amplifying the fusion fragment in a PCR2 system to obtain a large amount of fusion fragment amplification products;
a5, seamlessly linking the large amount of the fusion fragment amplification products obtained in step a4 with the pP43NMK linear plasmid obtained in step a3 to obtain a first resulting fusion plasmid; then mixing and incubating the first resulting fusion plasmid with Escherichia coli JM109 competent cells to obtain an E. coli JM109 containing the first resulting fusion plasmid;
a6, culturing the E. coli JM109 containing the first resulting fusion plasmid in a first LB culture medium containing ampicillin to obtain a first thalli, and then performing a plasmid extraction on the first thalli to obtain a second resulting fusion plasmid;
a7, adding a histidine label at a terminal of the flavoprotein reductase gene in the second resulting fusion plasmid obtained in step a6 to obtain an E. coli TOP10 containing a third resulting fusion plasmid;
a8, culturing the E. coli TOP10 containing the third resulting fusion plasmid obtained in step a7 in a second LB culture medium containing ampicillin to obtain a second thalli, and then performing the plasmid extraction on the second thalli to obtain a fourth resulting fusion plasmid.

4. The color former according to claim 3, wherein the method for preparing the fusion enzyme comprises: thawing B. subtilis 168 competent cells at a room temperature, and adding the fourth resulting fusion plasmid into thawed cells for an incubation, a culture, and a screening to obtain the fusion strain.

5. The color former according to claim 4, wherein the method for preparing the fusion enzyme comprises:
c1, inoculating a single colony in the fusion strain to a culture medium containing kanamycin to be cultured, and then isolating a strain from the culture medium containing the kanamycin;
c2, sufficiently washing the strain isolated in step c1 and then performing a wall breaking treatment on the strain, and then isolating to obtain a supernatant, to obtain a fusion bacterial crude extract; and
c3, mixing and incubating the fusion bacterial crude extract obtained in step c2 with the Ni-NTA agarose purification resin, and performing a protein elution to obtain a fusion enzyme solution; wherein a concentration of the fusion enzyme in the fusion enzyme solution is 0.1 mg/L-0.5 mg/L.

6. A preparation method of a color former, comprising using a fusion enzyme producing nitric oxide, wherein the fusion enzyme is formed by sequentially combining a nitric oxide synthase, a flavoprotein, and a flavoprotein reductase pairwise via linker peptides.

7. The color former according to claim 1, wherein the color former is used in a color formation of meat products, and the meat products comprise fermented and/or non-fermented meat products.

8. The color former according to claim 2, wherein the color former is used in a color formation of meat products, and the meat products comprise fermented and/or non-fermented meat products.

9. The color former according to claim 3, wherein the color former is used in a color formation of meat products, and the meat products comprise fermented and/or non-fermented meat products.

10. The color former according to claim 4, wherein the color former is used in a color formation of meat products, and the meat products comprise fermented and/or non-fermented meat products.

11. The color former according to claim 5, wherein the color former is used in a color formation of meat products, and the meat products comprise fermented and/or non-fermented meat products.

12. A color forming method of the color former according to claim 1, comprising mixing and incubating the color former with a substrate to achieve a color formation of the substrate;
wherein the substrate comprises a metmyoglobin or meat products.

13. The color forming method according to claim 12, wherein a method for preparing the fusion enzyme comprises:
providing a fusion plasmid at least containing a nitrogen oxide synthase gene, a flavoprotein gene, and a flavoprotein reductase gene;
transforming a Bacillus subtilis with the fusion plasmid to obtain a fusion strain; and
culturing the fusion strain, mixing and incubating the fusion strain subjected to wall breaking with a Ni-NTA agarose purification resin, and then isolating to obtain the fusion enzyme.

14. The color forming method according to claim 13, wherein the method for preparing the fusion enzyme comprises:
a1, in a polymerase chain reaction (PCR) system, amplifying a nitrogen oxide synthase gene fragment, a flavoprotein gene fragment, and a flavoprotein reductase gene fragment based on a B. subtilis 168 genomic DNA as a template; wherein an upstream primer of the nitric oxide synthase used in the PCR system has the sequence as shown in SEQ ID NO: 1, and a downstream primer of the nitric oxide synthase has the sequence as shown in SEQ ID NO: 2 or SEQ ID NO: 3; an upstream primer of the flavoprotein has the sequence as shown in SEQ ID NO: 4, and a downstream primer of the flavoprotein has the sequence as shown in SEQ ID NO: 5; an upstream primer of the flavoprotein reductase has the sequence as shown in SEQ ID NO: 6, and a downstream primer of the flavoprotein reductase has the sequence as shown in SEQ ID NO: 7;
a2, in the PCR system, amplifying a linearized plasmid in the PCR system based on a pP43NMK as a template to obtain a linearized pP43NMK plasmid;
a3, adding a FastDigest DpnI enzyme into the linearized pP43NMK plasmid obtained in step a2, and treating a resulting plasmid on a PCR instrument to eliminate a cyclic plasmid, and then inactivating the FastDigest DpnI enzyme to obtain a pP43NMK linear plasmid;
a4, in a PCR1 system, fusing the nitrogen oxide synthase gene fragment, the flavoprotein gene fragment, and the flavoprotein reductase gene fragment obtained in step a1 through a triple fusion PCR to obtain a fusion fragment, and then further amplifying the fusion fragment in a PCR2 system to obtain a large amount of fusion fragment amplification products;
a5, seamlessly linking the large amount of the fusion fragment amplification products obtained in step a4 with the pP43NMK linear plasmid obtained in step a3 to obtain a first resulting fusion plasmid; then mixing and incubating the first resulting fusion plasmid with Escherichia coli JM109 competent cells to obtain an E. coli JM109 containing the first resulting fusion plasmid;
a6, culturing the E. coli JM109 containing the first resulting fusion plasmid in a first LB culture medium containing ampicillin to obtain a first thalli, and then performing a plasmid extraction on the first thalli to obtain a second resulting fusion plasmid;
a7, adding a histidine label at a terminal of the flavoprotein reductase gene in the second resulting fusion plasmid obtained in step a6 to obtain an E. coli TOP10 containing a third resulting fusion plasmid;
a8, culturing the E. coli TOP10 containing the third resulting fusion plasmid obtained in step a7 in a second LB culture medium containing ampicillin to obtain a second thalli, and then performing the plasmid extraction on the second thalli to obtain a fourth resulting fusion plasmid.

15. The color forming method according to claim 14, wherein the method for preparing the fusion enzyme comprises: thawing B. subtilis 168 competent cells at a room temperature, and adding the fourth resulting fusion plasmid into thawed cells for an incubation, a culture, and a screening to obtain the fusion strain.

16. The color forming method according to claim 15, wherein the method for preparing the fusion enzyme comprises:
c1, inoculating a single colony in the fusion strain to a culture medium containing kanamycin to be cultured, and then isolating a strain from the culture medium containing the kanamycin;
c2, sufficiently washing the strain isolated in step c1 and then performing a wall breaking treatment on the strain, and then isolating to obtain a supernatant, to obtain a fusion bacterial crude extract; and
c3, mixing and incubating the fusion bacterial crude extract obtained in step c2 with the Ni-NTA agarose purification resin, and performing a protein elution to obtain a fusion enzyme solution; wherein a concentration of the fusion enzyme in the fusion enzyme solution is 0.1 mg/L-0.5 mg/L.

17. The color forming method according to claim 12, comprising: mixing the color former with the substrate to obtain a mixture, and incubating the mixture for 0.5 h-30 h at 4° C.-42° C.;
and/or, wherein a concentration of the fusion enzyme in the color former is 0.1 mg/mL-0.5 mg/mL.

18. The color forming method according to claim 12, comprising: adding the color former into an LB culture medium containing 4 mg/mL-6 mg/mL metmyoglobin and 8 mmol/L-12 mmol/L L-arginine, instantly covering a top of the LB culture medium with a sterile paraffin oil and performing an anaerobic incubation for 0.5 h-16 h at 4° C.-42° C.; wherein a volume ratio of the color former to the LB culture medium to the sterile paraffin oil is (100 μL-200 μL): (1 mL-5 mL): (100 μL-300 μL);
and/or, the color forming method comprises: evenly mixing the color former with the meat products and accessories to form a mixed minced meat and palletizing the mixed minced meat at 15° C.-42° C., covering a palletized minced meat with a plastic wrap to be placed for 6 h-30 h, and then storing and processing a resulting mixed minced meat; wherein the accessories comprise L-arginine, sodium chloride, and glucose; a mass ratio of the color former to the meat products is (8 mg-12 mg): 100 g; and a mass ratio of the meat products to the L-arginine to the sodium chloride to the glucose is 100: (0.5-1.0): (0.5-5.0): (0.5-10).
